(19) United States
(12) Patent Application Publication
(10) Pub. No.: US 2020/0384179 A1
(43) Pub. Date: Dec. 10, 2020

(54) DIALYSIS MACHINE HAVING SECURITY AGAINST CONFUSION FOR HYDRAULIC CONNECTORS

(71) Applicant: Fresenius Medical Care Deutschland GmbH, Bad Homburg (DE)

(72) Inventors: Jürgen HÄCKER, Neu-Anspach (DE); Oliver BOND, Sennfeld (DE); Winfried BREHM, Hofheim (DE)

(73) Assignee: Fresenius Medical Care Deutschland GmbH, Bad Homburg (DE)

(21) Appl. No.: 16/767,199

(22) PCT Filed: Nov. 26, 2018
(86) PCT No.: PCT/EP2018/082582

§ 371 Date: May 27, 2020

(30) Foreign Application Priority Data
Nov. 28, 2017 (DE) 10 2017 128 079.0

(51) Int. Cl. A61M 1/30; A61M 39/10

(52) U.S. Cl. CPC A61M 1/301; A61M 2205/58; A61M 39/10

(57) ABSTRACT

The invention relates to a dialysis machine having two hydraulic connectors at the dialyzate side to which a respective connection line having a filter coupling is connected for connection to a dialyzer, with the hydraulic connectors being an inflow connector for feeding fresh dialysis liquid to the dialyzer and an outflow connector for draining consumed dialyzate from the dialyzer, and having two parking positions associated with the respective hydraulic connectors for storing the filter couplings when not in operation, wherein the inflow connector and the parking position associated with the outflow connector are arranged spaced apart from one another at the machine and the length of the connection line connected to the inflow connector is smaller than this spacing, and wherein the outflow connector and the parking position associated with the inflow connector are arranged spaced apart from one another at the machine and the length of the connection line connected to the outflow connector is smaller than this spacing.

The invention relates to a dialysis machine having an inflow connector for feeding fresh dialysis liquid to the dialyzer, having an outflow connector for draining consumed dialyzate from the dialyzer, and having two parking positions respectively associated with one connector, wherein a security against confusion is provided that ensures a storing of the inflow connector and of the outflow connector at the respective associated parking positions.

The dialyzer interface is a fluidic interface of a dialysis machine on which increased hygienic demands are made. The dialyzer has two hydraulic connectors at the dialyzate side. One serves as the inflow for dialysis fluid, the other for leading off the consumed dialyzate. Consumed dialyzate is potentially infectious. When dismantling after a treatment, dialyzate moves onto the parking positions or parking connectors of the machine. Currently which connector is to be plugged to which parking position is indicated via a color code (for example, red to red and blue to blue). If a mix up arises here due to incorrect handling, there is a risk that the inflow coupling will be contaminated. On a subsequent treatment, pathogenic organisms could then move onto the hydraulic side of the dialyzer.

It is the object of the invention to provide a dialyzer with which such a confusion can be reliably precluded.

Against this background, the invention relates to a dialysis machine having two hydraulic connectors at the dialyzate side to which a respective connection line having a filter coupling is connected for connection to a dialyzer, with the hydraulic connectors being an inflow connector for feeding fresh dialysis liquid to the dialyzer and an outflow connector for draining consumed dialyzate from the dialyzer, and having two parking positions associated with the respective hydraulic connectors for storing the filter couplings when not in operation. Provision is made that the inflow connector and the parking position associated with the outflow connector are arranged spaced apart from one another at the machine and the length of the connection line connected to the inflow connector is smaller than this spacing and that the outflow connector and the parking position associated with the inflow connector are arranged spaced apart from one another at the machine and the length of the connection line connected to the outflow connector is smaller than this spacing.

It is thus not possible to incorrectly place the filter coupling of the inflow connector at the parking position associated with the outflow connector and vice versa. In accordance with the invention, the positions of the connectors and of the parking positions and the lengths of the connection lines are thus fixed in a suitable manner to prevent a storage of the filter couplings at incorrect parking positions.

Provision is made in an embodiment that the inflow connector is arranged at a first position and the outflow connector is arranged at a second position at the machine that is spaced apart from the first position, said spacing being larger than the length of both the connection line connected to the inflow connector and the connection line connected to the outflow connector. The spacing is, however, not necessarily larger than the sum of the lengths of both connection lines.

Provision is made in an embodiment that the inflow connector and the parking position associated with the inflow connector are arranged together at a first position at the machine and that the outflow connector and the parking position associated with the outflow connector are arranged together at a second position at the machine that is at a spacing from the first position that is larger than the length of both the connection line connected to the inflow connector and the connection line connected to the outflow connector. The connectors and the associated parking positions are therefore each arranged in direct proximity to the machine and form a common station. The stations are arranged at a spacing from one another that is selected such that neither the filter coupling of the inflow connector nor the filter coupling of the outflow connector can be placed at the incorrect parking position.

Provision is made in an embodiment that the dialysis machine has a holder for a dialyzer that is configured and is arranged at the machine such that a dialyzer placed in the holder is spatially disposed between the inflow connector and the outflow connector. This means that the spacing of the dialyzer from both the inflow connector and the outflow connector is smaller than the spacing between the inflow connector and the outflow connector. Both filter couplings can thus be connected to the dialyzer despite the limited lengths of the connection lines.

The invention further relates to an apparatus comprising a dialysis machine in accordance with the invention and a dialyzer that is placed into a holder of the dialysis machine provided for this purpose and that has an input for fresh dialysis liquid and an output arranged at a spacing therefrom for consumed dialyzate, with the spacing of the input from the outflow connector being greater than the length of the connection line connected to the outflow connector and with the spacing of the output from the inflow connector being greater than the length of the connection line connected to the inflow connector.

A connection of the filter couplings to the incorrect position at the dialyzer can thus furthermore be prevented. The spacing of the input from the inflow connector is naturally smaller than the length of the connection line connected to the inflow connector and the spacing of the output from the outflow connector is larger than the length of the connection line connected to the outflow connector so that a correct connection can take place.

Further details and advantages of the invention result from the following embodiment described with reference to the Figures. There are shown in the Figures:

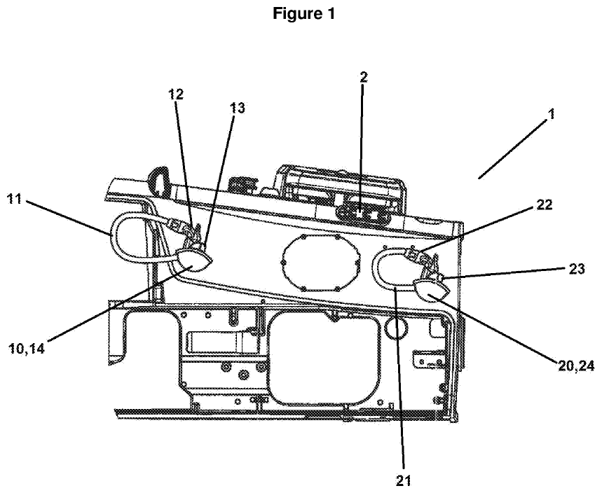

FIG. 1: a view of a dialysis machine in accordance with the invention, with the filter couplings of the hydraulic connectors being stored in the respective parking positions;

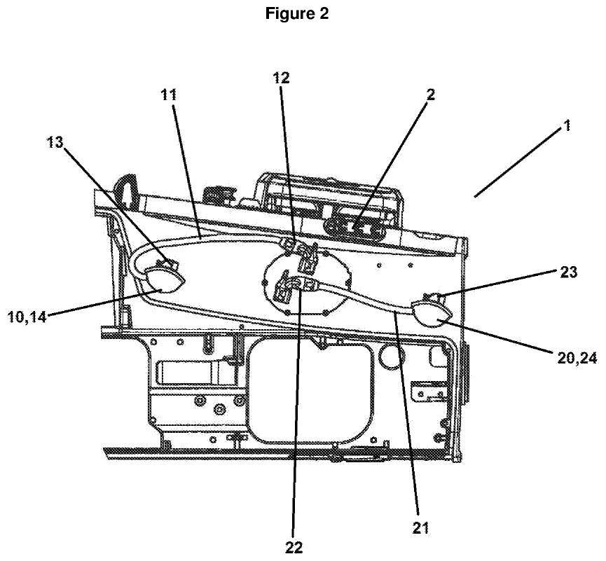

FIG. 2: a view of the same dialysis machine, with the filter couplings of the hydraulic connectors being removed from the respective parking positions;

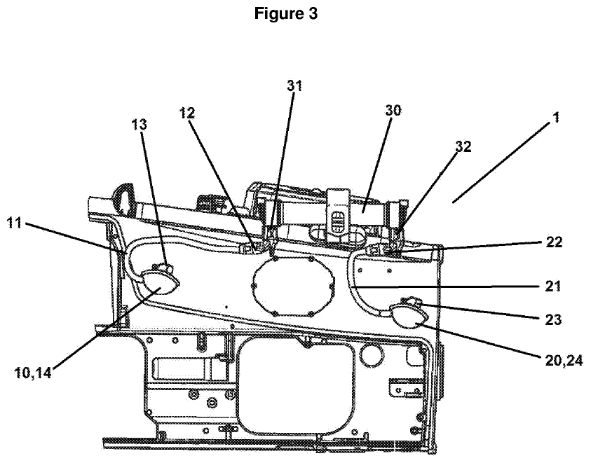

FIG. 3: a view of a dialysis machine in accordance with the invention with an inserted dialyzer, with the filter couplings of the hydraulic connectors not being coupled to the dialyzer; and

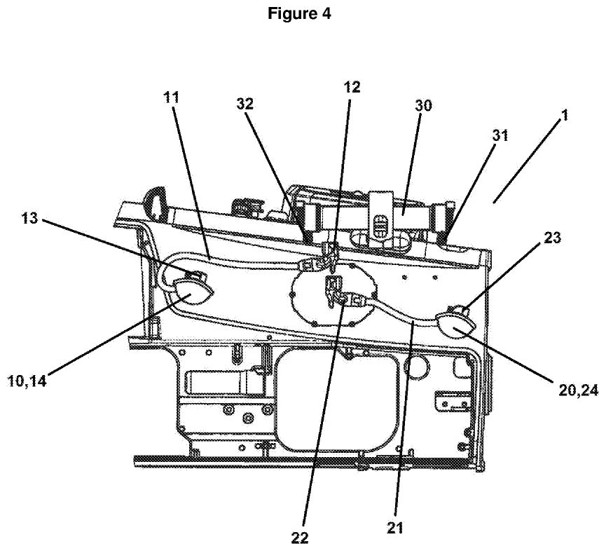

FIG. 4: a view of the same dialysis machine with an inserted dialyzer, with the filter couplings of the hydraulic connectors being coupled to the dialyzer.

The Figures show a dialysis machine 1 in accordance with the invention that comprises an inflow connector 10 for feeding fresh dialysis liquid to a dialyzer 30 and an outflow connector 20 for draining consumed dialyzate from the dialyzer 30.

The inflow connector 10 is provided with a first connection line 11 having a first filter coupling 12. The outflow connector 20 is provided with a second connection line 21 having a first filter coupling 22. The machine 1 furthermore comprises a first parking position 13 for the first filter coupling 12 of the inflow connector 10 and a second parking position 23 for the second filter coupling 22 of the outflow connector 20. The inflow connector 10 and the first parking position 13 form a common inflow station 14 at the machine 1. The outflow connector 20 and the second parking position 23 form a common outflow station 24 at the machine 1.

The inflow station 14 and the outflow station 24 are arranged at a spacing from one another that is larger than the length of both the first connection line 11 and the second connection line 21. It is therefore impossible to place the first filter coupling 12 at the second parking position 23 and is it also impossible to place the second filter coupling 22 at the first parking position 13, as can be recognized in FIG. 2. It is only possible to place the first filter coupling 12 at the first parking position 13 and the second filter coupling 22 at the second parking position 23, as can be recognized in FIG. 1.

The dialysis machine 1 furthermore comprises a holder 2 for locking a dialyzer 30 in a defined position. The dialyzer 30 comprises an inlet 31 for fresh dialysis liquid arranged at one end of the dialyzer 30 and an outlet 32 for consumed dialyzate arranged at the oppositely disposed end of the dialyzer 30.

The holder 2 is disposed spatially between the inflow station 14 and the outflow station 24, which means that the spacing of the dialyzer from both the inflow station 14 and from the outflow station 24 is smaller than the spacing between the inflow station 14 and the outflow station 24. Both filter couplings 12 and 22 can thus be connected to the dialyzer despite the limited lengths of the connection lines 11 and 21, as is shown in FIG. 3. However, the position of the dialyzer 30 in the holder 2 and the location of the inlet 31 and of the outlet 32 at the dialyzer are selected such that the spacing of the inlet 31 from the outflow station 24 is larger than the length of the connection line 21 connected to the outflow connector 20 and such that the spacing of the outlet 32 from the inflow station 14 is larger than the length of the connection line 11 connected to the inflow connector 10, as is shown in FIG. 4.

1. A dialysis machine having two hydraulic connectors at the dialyzate side to which a respective connection line having a filter coupling is connected for connection to a dialyzer, with the hydraulic connectors being an inflow connector for feeding fresh dialysis liquid to the dialyzer and an outflow connector for draining consumed dialyzate from the dialyzer, and having two parking positions associated with the respective hydraulic connectors for storing the filter couplings when not in operation,
characterized in that
the inflow connector and the parking position associated with the outflow connector are arranged spaced apart from one another at the machine and the length of the connection line connected to the inflow connector is smaller than this spacing and that the outflow connector and the parking position associated with the inflow connector are arranged spaced apart from one another at the machine and the length of the connection line connected to the outflow connector is smaller than this spacing.

2. A dialysis machine in accordance with claim 1, characterized in that the inflow connector is arranged at a first position and the outflow connector is arranged at a second position at the machine that is spaced apart from the first position, said spacing being larger than the length of both the connection line connected to the inflow connector and the connection line connected to the outflow connector.

3. A dialysis machine in accordance with claim 2, characterized in that the inflow connector and the parking position associated with the inflow connector are arranged together at a first position at the machine and that the outflow connector and the parking position associated with the outflow connector are arranged together at a second position at the machine that is at a spacing from the first position that is larger than the length of both the connection line connected to the inflow connector and the connection line connected to the outflow connector.

4. A dialysis machine in accordance with claim 1, characterized in that the dialysis machine has a holder for a dialyzer that is configured and is arranged at the machine such that a dialyzer placed in the holder is spatially disposed between the inflow connector and the outflow connector.

5. An apparatus comprising a dialysis machine in accordance with claim 4 and a dialyzer that is placed in the holder and that has an input for fresh dialysis liquid and an output arranged at a spacing therefrom for consumed dialyzate, with the spacing of the input from the outflow connector being greater than the length of the connection line connected to the outflow connector and with the spacing of the output from the inflow connector being greater than the length of the connection line connected to the inflow connector.
